(19) United States
(12) Patent Application Publication
(10) Pub. No.: US 2013/0197084 A1
(43) Pub. Date: Aug. 1, 2013

(54) NUTRIENT SENSOR

(71) Applicant: BAYLOR RESEARCH INSTITUTE, Dallas, TX (US)

(72) Inventor: Charles R. Roe, Rockwall, TX (US)

(73) Assignee: BAYLOR RESEARCH INSTITUTE, Dallas, TX (US)

(21) Appl. No.: 13/794,493

(22) Filed: Mar. 11, 2013

Related U.S. Application Data
(63) Continuation of application No. 11/862,081, filed on Sep. 26, 2007, now abandoned.
(60) Provisional application No. 60/847,252, filed on Sep. 26, 2006.

(51) Int. Cl. A61K 31/20

(52) U.S. Cl. CPC A61K 31/20
USPC 514/547; 514/558; 514/557

(57) ABSTRACT

The present invention includes compositions and methods for treating the effects of catabolism in a patient by providing the patient with an amount of an odd-chain fatty acid sufficient to increase the intracellular ratio of AMP to ATP and reduce the activity of AMPK.

### CROSS-REFERENCE TO RELATED APPLICATIONS

This application claims priority to U.S. Provisional Application Ser. No. 60/847,252 filed on Sep. 26, 2006. the entire contents of which are incorporated herein by reference.

### TECHNICAL FIELD OF THE INVENTION

The present invention relates in general to the field of nutrient sensors and intracellular metabolism, and more particularly, to the use of odd-chain fatty acids to modulate the activity of AMP-activated Protein Kinase (AMPK) to increase or decrease the rate of cellular catabolism.

### BACKGROUND OF THE INVENTION

Without limiting the scope of the invention, its background is described in connection with intracellular metabolism.

Since the early discoveries in cellular metabolism, biochemistry and the identification of the genes that code for the critical enzymes involved in metabolism, dietary therapy for inborn errors of metabolism has focused primarily on the restriction of the precursor to an affected metabolic pathway. These early discoveries have led to numerous complementary therapies in which missing precursors or nutrients are provided in the diet alone or in combination with one or more pharmaceutical drugs.

Cellular metabolism has two distinct divisions: anabolism, in which a cell uses energy to build complex molecules and perform other life functions such a creating cellular structure; and catabolism, in which a cell breaks down complex molecules to yield energy and reducing power. Cell metabolism involves extremely complex sequences of controlled chemical reactions, control and regulatory mechanisms, feedback loops and the increase and decrease of gene expression.

Despite years of nutritional and drug therapies, there exists a need for improvements in the energy processing and metabolism of cells at the micro and macro levels. Often, existing therapies have focused on the precursors for metabolism, rather than the control mechanisms of metabolism.

### SUMMARY OF THE INVENTION

The present invention is based on the recognition that comprehensive therapies for numerous unrelated diseases have common control regulatory mechanisms. Anaplerotic therapy is based on the concept that an energy deficit in inborn diseases might be improved by providing alternative substrate for both the citric acid cycle (CAC) and the electron transport chain for enhanced ATP production. One critical regulatory component is the AMP-activated Protein Kinase (AMPK).

The present invention includes compositions and methods for treating the effects of catabolism in a patient by providing the patient with an amount of an odd-chain fatty acid sufficient to increase the intracellular ratio of adenosine monophosphate (AMP) to adenosine triphosphate (ATP) and reduce the activity of the AMP-activated Protein Kinase (AMPK). The odd-chain fatty acid may be heptanoate, pentanoate, triheptanoate, tripentanoate and combinations thereof. The odd-chain fatty acid may even be able to reduce the activity of the mammalian target of rapamycin (mTOR); the activity of mTOR may also be used to detect the effect of the compositions and methods of the present invention. The odd-chain fatty acid is generally metabolized to increase the intracellular levels of ADP or ATP, thereby turning off intracellular AMPK.

As such, providing the patient with the odd-chain fatty acid serves to turn on and off the nutrient switch, AMPK, which is responsible for directing the biochemical switching between anabolism and catabolism. The present invention takes advantage of odd-chain fatty acids to circumvent or shunt the regular biochemical pathways to reach the switching mechanism itself, namely changes or modulation of the relative concentrations of AMP, adenosine diphosphate (ADP) and ATP. For example, the odd-chain fatty acid reduces cellular catabolism by increasing the levels of ATP, thereby turning off AMPK. Depending on the generally activation state of a patient or organ the activity of AMPK may be modulated by, e.g., providing the patient or organ with between about 1 and about 40%, or between 20 and 35% of the daily dietary caloric requirement for the patient in odd-chain fatty acids. The skilled artisan will recognize that the patient or their organ may receive the odd-chain fatty acid though a variety or methods and location. Non-limiting example of methods of providing the patient the odd-chain fatty acid include orally, enterally, parenterally, nasally, intravenously or combinations thereof, and the like.

The present invention also include a method for treating the reducing intracellular catabolism in a patient in need thereof by providing the patient with an amount of an odd-chain fatty acid sufficient to increase the intracellular ratio of AMP to ATP. The odd-chain fatty acid may be heptanoate, pentanoate, triheptanoate, tripentanoate and combinations thereof.

Yet another method of the present invention includes compositions and methods for modulating intracellular metabolism in a patient in need thereof by determining the metabolic state of a patient by identifying the level of activation of AMPK; and changing the percentage of an odd-chain fatty acid in the patient's diet to change the intracellular ratio of AMP to ATP and the activation state of the AMPK. Again, the odd-chain fatty acid may include heptanoate, pentanoate, triheptanoate, tripentanoate and combinations thereof.

Another embodiment of the present invention includes compositions for modulating the activity of intracellular AMPK that include a nutritionally effective amount of an odd-chain fatty acid that is sufficient to change the intracellular activity of AMPK to increase or decrease the amount of intracellular catabolism. The nutritionally effective amount of an odd-chain fatty acid may be heptanoate, pentanoate, triheptanoate, tripentanoate and combinations thereof, and may be between 0.01 and 40 percent of a patient's daily dietary caloric requirement. The composition of the present invention may be provided in any of a wide variety of dosage forms, alone or in combination with a carrier, excipients, stabilizers, potentiators, solubilizers, preservatives and the like. The composition may even include one or more lipids, carbohydrates, proteins, saccharides, amino acids, vitamins, minerals, metals and combinations thereof. The odd-chain fatty acid may be formulated for oral, enteral, parenteral, intravenous, subcutaneous, transcutaneous delivery or combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 summarizes the hepatic metabolism of heptanoate (C7) and the enzymes that are required for its metabolism. The steps of β-oxidation representing potential fat oxidation disorders are also outlined. Heptanoate not only provides fuel to the citric acid cycle (CAC) in liver, but also produces 5-carbon ‘ketone’ bodies for export to other organs for fuel (propionyl-CoA and acetyl-CoA) for the CAC, thus providing an energy source in all organs (BHP=β-hydroxypentanoate; BKP=β-ketopentanoate).

FIG. 2 summarized the metabolic abnormalities observed in type B pyruvate carboxylase deficiency. The deficit of oxaloacetate (OAA) limits aspartate required for the conversion of citrulline to argininosuccinate in the urea cycle. The cytosolic ratio of NADH:NAD shifts pyruvate to lactate, while the decreased production of NADH via the CAC lowers that ratio and permits acetoacetate to accumulate rather than being converted to 3-hybroxybutyrate. These changes reflect the altered redox states in both the cytosol and the mitochondria in the liver.

FIG. 3 summarizes the biochemical pathways for production and unidirectional export of alanine from skeletal muscle to liver as a source of pyruvate for hepatic mitochondria. (alanine cycle) in acid maltase deficiency. Abbreviations: MDH (malate dehydrogenase), PK (pyruvate kinase), AAT (alanine aminotransferase), ME (malic enzyme).

FIG. 4 summarizes the biochemical pathway for metabolism of heptanoate and the production and export of the 5-carbon ketone body (BHP) in liver and BHP utilization in skeletal muscle in acid maltase deficiency. Heptanoate reduces the need for muscle alanine by fuelling the CAC in both organ systems. Abbreviations: same as in FIG. 4, plus SCOT (succinyl-CoA transferase).

FIG. 5 is a summary of the activation of the nutrient sensors AMPK and mTOR. Consequences for intermediary metabolism (catabolism vs synthesis) and the anaplerotic role of heptanoate.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “subject” or “patient” are intended to include living organisms that may have one or more conditions generally referred to as polysaccharide storage diseases. Examples of subjects include humans, monkeys, horses, cows, sheep, goats, dogs, cats, mice, rats, and transgenic species thereof. Other examples of subjects include experimental animals such as mice, rats, dogs, cats, goats, sheep, pigs, and cows. A subject can be a human suffering from, or suspected of having a catabolic energy state, wasting (e.g., cachexia), in need of energy for survival or even for enhancing performance or general nutrition. Depending on the nature of the deficiency (acute versus chronic), disease state (cancer, cachexia, inherent error in metabolism, acquired metabolic errors, etc.), nutritional condition and the like, the present invention may be used to treat one or more of those conditions in which the patient is in need of controlling the anabolic and/or catabolic state of cells, e.g., organs or the entire patient.

As used herein, the phrases “therapeutically effective dosage” or “therapeutically effective amount” is an amount of a compound or mixtures of compounds, such as the odd chain fatty acids and precursors or derivatives thereof, that reduce the amount of one or more symptoms of the condition in the infected subject by at least about 20%, at least about 40%, even more at least about 60%, 80% or even 100% relative to untreated subjects. Active compounds are administered at a therapeutically effective dosage sufficient to treat a condition associated with a condition in a subject. For example, the efficacy of a compound can be evaluated in patients or animal model systems that may be predictive of efficacy in treating the disease in humans or animals.

As used herein the term, “essential fatty acids” is used to describe fats and oils in foods are made up of basic units called fatty acids. The term, “odd-chain fatty acids” is used to describe fats and oils in foods are made up of an odd-number of carbons in the fatty chain. In the body, fatty acid chains typically travel in attached to glycerol, a “triglyceride.” Based on their chemical structure, fatty acids are classified into 3 major categories: monounsaturated, polyunsaturated, or saturated fats. The oils and fats that people and animals eat are nearly always mixtures of these 3 types of fatty acids, with one type predominating. Two specific types of polyunsaturated fatty acids, linoleic and alpha-linolenic, are called essential fatty acids. They must be present in the diet in adequate amounts because they are considered necessary for proper nutrition and health. Linoleic acid (LA) is an omeaga-6 fatty acid and is found in many oils, e.g., corn, safflower, soybean and sunflower, whole grains and walnuts. Alpha-linolenic acid (ALA) is a plant precursor of docosahexanoic acid (DHA). Sources of ALA include seaweeds and green leaves of plants (in very small amounts), soybeans, walnuts, butternuts, some seeds (flax, chia, hemp, canola) and the oils extracted from these foods.

As used herein, the term “nutritionally effective amount” is used to mean the amount of odd chain fatty acids that will provide a beneficial nutritional effect or response in a mammal For example, as with a nutritional response to vitamin- and mineral-containing dietary supplements varies from mammal to mammal, it should be understood that nutritionally effective amounts of the odd chain fatty acids will vary. Thus, while one mammal may require a particular profile of vitamins and minerals present in defined amounts, another mammal may require the same particular profile of vitamins and minerals present in different defined amounts. Such is the case with the nutritionally effective amounts of the odd chain fatty acids of the present invention, in Which the supplementation may be used to add C3 and C2 carbon chains into the liver and/or the heart, muscle, brain and kidney.

When provided as a dietary supplement or additive, the odd chain fatty acids of the invention has been prepared and administered to mammals in powdered, reconstitutable powder, liquid-solid suspension, liquid, capsule, tablet, caplet, lotion and cream dosage forms. The skilled artisan in the science of formulations can use the odd chain fatty acids disclosed herein as a dietary supplement that may be formulated appropriately for, e.g., irrigation, ophthalmic, otic, rectal, sublingual, transdermal, buccal, vaginal, or dermal administration. Thus, other dosage forms such as chewable candy bar, concentrate, drops, elixir, emulsion, film, gel, granule, chewing gum, jelly, oil, paste, pastille, pellet, shampoo, rinse, soap, sponge, suppository, swab, syrup, chewable gelatin form, chewable tablet and the like, can be used.

Due to varying diets among people, the dietary odd chain fatty acids of the invention may be administered in a wide range of dosages and formulated in a wide range of dosage unit strengths. It should be noted that the dosage of the dietary supplement can also vary according to a particular ailment or disorder that a mammal is suffering from when taking the supplement. For example, a person suffering from chronic fatigue syndrome or fibromyalgia will generally require a dose different than an athlete wanting to attain a nutritional benefit. An appropriate dose of the dietary supplement can be readily determined by monitoring patient response, i.e., general health, to particular doses of the supplement. The appropriate doses of the supplement and each of the agents can be readily determined in a like fashion by monitoring patient response, i.e., general health to particular doses of each.

The odd chain fatty acids may be administered simultaneously or sequentially in one or a combination of dosage forms. While it is possible and even likely that the present dietary supplement will provide an immediate overall health benefit, such benefit may take days, weeks or months to materialize. Nonetheless, the present dietary odd chain fatty acid supplement will provide a beneficial nutritional response in a mammal consuming it.

The odd-chain fatty acids of the present invention may be administered, e.g., orally or by subcutaneous, intravenous, intraperitoneal, etc., administration (e.g. by injection). Depending on the route of administration, the active compound may be neutralized, made miscible, at least partially or fully water-soluble or even coated in a material to protect the odd-chain fatty acids from the action of bases, acids, enzymes or other natural conditions that may interfere with their effectiveness, uptake or metabolic use.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, emulsifiers, liposomes, or a diluent. Pharmaceutically acceptable diluents include saline and aqueous buffer solutions. The therapeutic odd-chain fatty acids may be dispersed in glycerol, liquid polyethylene glycols, and mixtures thereof and in oils. Under ordinary conditions of storage and use, these preparations may contain a preservative to prevent the growth of microorganisms.

Pharmaceutical compositions that include the odd-chain fatty acids of the present invention suitable for injectable use may include sterile aqueous solutions, dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

The odd-chain fatty acids may be provided with a carrier in a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, fur example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The odd-chain fatty acids may be provided in one or more controlled sizes and characteristics with one or more water-soluble polymers depending on the size and structural requirements of the patient, e.g., the particles may be small enough to traverse blood vessels when provided intravenously. Either synthetic or naturally occurring polymers may be used, and while not limited to this group, some types of polymers that might be used are polysaccharides (e.g. dextran, ficoll), proteins (e.g. poly-lysine), poly(ethylene glycol), or poly(methacrylates). Different polymers, because of their different size and shape, will produce different diffusion characteristics for the odd-chain fatty acids in the target tissue or organ.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, methods of preparation include: vacuum drying, spray freezing, freeze-drying and the like, which yield a powder of the active ingredient (i.e., the therapeutic compound) plus any additional desired ingredient from a previously sterile-filtered solution thereof.

The odd-chain fatty acids can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. The odd-chain fatty acids may be incorporated with excipients and used in the form of ingestible tablets, buccal tablets, troches, capsules, elixirs, suspensions, syrups, wafers, and the like. The amount of odd-chain fatty acids in the compositions and preparations may, of course, be varied depending on, e.g., the age, weight, gender, condition, disease and course of treatment of the individual patient. Pediatric doses are likely to differ from adult doses as will be known to the skilled artisan. The amount of the therapeutic compound in such therapeutically useful compositions is such that a suitable dosage will he obtained.

A dosage unit for use with the odd chain fatty acids disclosed herein may he a single compound or mixtures thereof with other compounds, e.g., amino acids, nucleic acids, vitamins, minerals, pro-vitamins and the like. The compounds may be mixed together, form ionic or even covalent bonds. For pharmaceutical purposes the odd chain fatty acids (e.g., C5, C7 and C15) of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may he used to provide the odd chain fatty acids of the present invention to a patient in need of therapy that includes a number of conditions, e.g., polysaccharide storage diseases, fatigue, low energy, wasting and the like. The odd chain fatty acids may also be administered as any one of known salt forms.

The total daily amount of odd chain fatty acids will vary depending on the condition and needs of a patient. For example, the odd chain fatty acids may be provided as a supplemental source of immediate, short-term, mid-term or long-term energy and may be provided in formulations that are immediately available, slow release or extended release. The dosage amount may he measured in grams per day, as a percentage of kCalories consumed in a day, as a percentage of the total daily caloric intake, as part of a fixed, a modified or a diet that changes over time. For example, a patient may need immediate intervention that “spikes” the amount of odd chain fatty acids to approach or reach ketosis. These “ketogenic” odd chain fatty acids will then be varied to not have other side effects, e.g., start with 40% of total caloric intake per day and then reduced over time as the patient's condition, symptoms, clinical course and/or metabolic conditions improves. The range of percentage caloric intake may vary from between about 0.01, 0.1, 1, 2, 5, 10, 15, 20, 22, 25, 30, 35, 40 or even higher percent, which may include one or more of the odd chain fatty acids (e.g., C5, C7 or C15 (available from, e.g., Sassol, Germany). One way to measure the effect and/or dosing of the odd chain fatty acids is to measure the amount that is detectable in body solids or fluids, e.g., biopsies and blood, respectively. A wide variety of odd chain fatty acids metabolites may be detected from multiple sources, e.g., urine, tears, feces, blood, sweat, breath and the like.

For example, when using C7 as the source of odd chain fatty acids these can be provided in the form of a triglyceride, e.g., tri-heptanoin. The triglyceride triheptanoin is provided in a concentration sufficient to provide a beneficial effect is most useful in this aspect of the present invention. The seven-carbon fatty acid may be provided, e.g.:

| | | | | |
|---|---|---|---|---|
| Infants | 1-4 | g/kg | 35% | kcalories |
| Children | 3-4 | g/kg | 33-37% | kcalories |
| Adolescent | 1-2 | g/kg | 35% | kcalories |
| Adults | 0.1-2 | g/kg | 35% | kcalories |

Goals have been set using 4 g/kg (within ideal body weight (IBW) range) for infants, children, and some adolescents. Goals have been set using 2 g/kg (within IBW range) for adolescents. Goals have been set using 2 g/kg (within IBW range) for adults; but toleration is 1-1.2 g per kg (which is 35% kcal of estimated needs).

The odd chain fatty acids are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the odd chain fatty acids may be formulated to provide, e.g., maximum and/or consistent dosing for the particular form for oral, rectal, topical, intravenous injection or parenteral administration. While the odd chain fatty acids may be administered alone or pure, they may also be provided as stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions of each incorporated herein by reference.

Odd chain fatty acids may be administered in the form of an emulsion and/or liposome, e.g., small unilamellar vesicles, large unilamallar vesicles and multilamellar vesicles, whether charged or uncharged. Liposomes may include one or more: phospholipids (e.g., cholesterol), stearylamine and/or phosphatidylcholines, mixtures thereof, and the like. Examples of emulsifiers for use with the present invention include: Imwitor 370, Imwitor 375, Imwitor 377, Imwitor 380 and Imwitor 829.

The odd chain fatty acid vesicles may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylaspartamidephenol, or polyethyleneoxide-polylysine substituted with palmitoyl residues, mixtures thereof, and the like. Furthermore, the vesicles may be coupled one or more biodegradable polymers to achieve controlled release of the odd chain fatty acids. Biodegradable polymers for use with the present invention include, e.g., polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the odd chain fatty acid in its native state. For oral administration in a liquid dosage form, the oral drug components may be combined with any oral, non-toxic, pharmaceutically acceptable inert carrier such as an emulsifier, a diluent or solvent (e.g., ethanol), glycerol, water, and the like. Examples of suitable liquid dosage forms include oily solutions or suspensions in water, pharmaceutically acceptable fats and oils, alcohols or other organic solvents, including esters, emulsions, syrups or elixirs, suspensions, solutions and/or suspensions reconstituted from non-effervescent granules and even effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in multiple editions of Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the odd chain fatty acids may also be delivered as an intranasal form via use of a suitable intranasal vehicle. For dermal and transdermal delivery, the odd chain fatty acids may be delivered using lotions, creams, oils, elixirs, serums, transdermal skin patches and the like, as are well known to those of ordinary skill in that art. Parenteral and intravenous forms may also include pharmaceutically acceptable salts arid/or minerals and other materials to make them compatible with the type of injection or delivery system chosen, e.g., a buffered, isotonic solution.

To the extent that the odd chain fatty acids may be made into a dry powder or form, they may be included in a tablet. Tablets will generally include, e.g,, suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. The odd chain fatty acids may be dissolved in an oil, e.g., a digestible oil such as soybean oil, cottonseed oil or olive oil. Non-digestible oils may also be used to have better control over the total caloric intake provided by the oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet, appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring sufficient active ingredient in deionized water and mixed with, e.g., up to 10% by volume propylene glycol, salts and/or water to deliver a composition, whether in concentrated or ready-to-use form. Given the nature of the odd chain fatty acids (alone, partially or fully-soluble in water) the amount and final concentration of the odd chain fatty acids may be varied such that the liquid may be provided intravenously using syringes and/or standard intravenous liquids or fluids. The solution will generally be made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

Mini-tablets. For mini-tablets, the active ingredient is compressed into a hardness in the range 6 to 12 Kp. The hardness of the final tablets is influenced by the linear roller compaction strength used in preparing the granulates, which are influenced by the particle size of e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for providing an immediate source of alternative cellular energy, e.g., before, during or after surgery. The dosage will generally be prepared sterile and ready-to-use, e.g., one or more containers that may be broken (e.g., sealed glass ampoules), pierced with a syringe for immediate administration or even a pressurized container. Such kits may further include, if desired, one or more of various conventional pharmaceutical kit components, such as, for example, containers with one or more pharmaceutically acceptable diluents, carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Pharmaceutical Dosage Forms. The odd chain fatty acids of the present invention may be provided in liquid form or may also be provided in a capsule, gelcap or other encapsulated form. Generally, one composition of the present invention is prepared by adding, e.g., half of the Kaolin clay or other carrier into the blended followed by addition of a first active salt form, e.g., the salt form that is less soluble in the final liquid suspension, e.g., as an emulsion in water. This process is particularly suitable for very large mixtures, e.g., 500, 1,000, 3,000 or even 5,000 liters.

One particular method of delivery of the odd chain fatty acids of the present invention is in a tablet, capsule or gelcap that is coated for enteric delivery. Enteric coating relates to a mixture of pharmaceutically acceptable excipients that is applied to, combined with, mixed with or otherwise added to a carrier to deliver the medicinal content, in this case one or more odd chain fatty acids (e.g., C5, C7, C11, C15, mixtures and combinations thereof) through the stomach unaltered for delivery into the intestines. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality. Methods for enteric coating are well known in the art.

Remington's Pharmaceutical Sciences, discloses that enteric polymer carries generally include carboxyl groups and hydrophobic groups in the molecule and the enteric polymer is dissolved in a solvent having a specific pH value through the dissociation of the carboxyl groups. For instance, commercially available hydroxypropylmethyl cellulose acetate succinate is a derivative of hydroxypmpylmethyl cellulose which is substituted with carboxyl groups (succinoyl groups) and hydrophobic groups (acetyl groups). Alginic acid, sodium alginate other natural materials may also be used to provide an enteric coating,

Other additives and excipients may then be added to the formulation of the partially water soluble carrier-active odd chain fatty acids mixture, e.g., adding Povidone (e.g., Povidone 30), Xantham gum (or other gums) and Sorbitol to a mixture of Kaolin Clay to provide a specific example of one formulation of the present invention. As will be apparent to those of skill in the art, the actual amount of the partially-excipient soluble active salt (e.g., non or partially water soluble) may be varied in accordance with the dissolution characteristics of the active, which may be further varied by addition of agents that affect the solubility and/or dissolution of the active in, e.g., water. As regards a pediatric formulation, the amount of active may be reduced in accordance with the dosage form approved for pediatric use.

One example of a liquid odd chain fatty acid(s) pharmaceutical composition may be prepared with the following components:

| Components | Weight | |
|---|---|---|
| Odd chain fatty acid(s) | 1.0 | Kg |
| emulsifier (e.g., Imwitor 375) | 100 | gr |
| Purified water (USP) | 2.0 | Kg |

The formulation may further include, e.g.:

| | | |
|---|---|---|
| Glycerin (USP) | 500.0 | ml |
| Sorbitol Solution, 70% (USP) | 500.0 | ml |
| Saccharin Sodium (USP) | 10.0 | gr |
| Citric Acid (USP) | 10.0 | gr |
| Sodium Benzoate (NF) | 6.0 | gr |
| Kollidon 30 | 330.0 | gr |
| Xanthan Gum 200 Mesh | 20.0 | gr |
| Bubble Gum Flavor | 11.1 | gr |
| Methylparaben | 1.0 | gr |
| Proplyparaben | 100 | mg |
| Propylene Glycol (USP) | 75 | ml |

| | |
|---|---|
| Additional ddH2O | QS to 5 liters. |

With appropriate increases of the above for scale-up.

A batch of mixed release odd chain fatty acids in an enveloped preparation on a carrier, e.g., beads, may be prepared with the following components:

| Components | Weight |
|---|---|
| Emulsified odd chain fatty acids | 8.0 mg |
| Carrier | 51.7 mg |
| Calcium Stearate | 4.0 mg |
| Talc | 4.0 mg |
| Pharmaceutical Glaze | 5.5 mg |

When combining odd chain fatty acids (C5, C7 and/or C15), these may be formulated as follows. A capsule for extended release of a first active and extended release of a second active in an enveloped formulation, in a single capsule:

| First Bead | Weight | | Second Bead | Weight | |
|---|---|---|---|---|---|
| odd chain fatty acid | 6.0 | mg | odd chain fatty acid | 2.0 | mg |
| C7 | | | C15 | | |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |

| | |
|---|---|
| Capsule | 1 |

When combining the odd chain fatty acids, these may be formulated as follows. A capsule for extended release of a first active and extended release of a second active in an enveloped formulation, in a single capsule:

| First Bead | Weight | | Second Bead | Weight | |
|---|---|---|---|---|---|
| odd chain fatty acid | 6.0 | mg | odd chain fatty acids | 2.0 | mg |
| C5 | | | C7 | | |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |

| | |
|---|---|
| Mini-capsule | 1 |

A formulation for extended release of odd chain fatty acids of a second active in an enveloped formulation, in a gelcap:

| Component | Weight | | Component | Weight | |
|---|---|---|---|---|---|
| odd chain fatty acid | 6.0 | mg | odd chain fatty acid | 2.0 | mg |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |

| | |
|---|---|
| Gelcap | 1 |

A formulation for rectal release of odd chain fatty acids in a suppository:

| Component | Weight | |
|---|---|---|
| Odd chain fatty acids | 100 | mg |
| Carrier | 10 | mg |
| Talc | 12.6 | mg |
| Calcium Stearate | 12.6 | mg |
| beeswax/glycerol | 1-2 | gr |

An enteric-coated soft gelatin capsule that includes the odd chain fatty acids (with or without an emulsifier) is made by coating the odd chain fatty acids with a lipophilic material to obtain granules, mixing the granules obtained in step with an oily matrix, antioxidants and preservatives to form a lipid suspension, mixing the lipid suspension within a soft gelatin film, and coating the soft gelatin film to obtain an enteric coated soft gelatin capsule.

The odd chain fatty acid(s), stearic acid and triethanolamine are heated and mixed to form an emulsified fluid. The resulting emulsified fluid is mixed well by a homogenizer to obtain an emulsified suspension and enterically coated. Examples of formulations include:

| Component | Weight |
|---|---|
| Odd Chain Fatty Acids | 360.0 g |
| Stearic acid | 78.6 g |
| Ethanolamine | 21.4 g |

| Component | Weight |
|---|---|
| Odd Chain Fatty Acids | 360.0 g |
| Stearic acid | 30.0 g |
| Triethanolamine | 20.0 g |

| Component | Weight |
|---|---|
| Odd Cham Fatty Acids | 400.0 g |
| Stearic acid | 77.0 g |
| Ethanolamine | 23.0 g |
| Cetyl alcohol | 50.0 g |

| Component | Weight |
|---|---|
| Odd Chain Fatty Acids | 245.0 g |
| Stearic acid | 38.5 g |
| Ethanolamine | 11.5 g |
| Cetyl alcohol | 50.0 g |
| Carboxymethyl cellulose | 25.0 g |

RECOMMENDED DAILY NUTRIENT INTAKE RANGES

| | | NUTRIENT | | | |
|---|---|---|---|---|---|
| AGE | | Protein | Energy | Fluid | C 7 |
| | | % of energy | kcal/kg/day | mL/kg | % Kcal/d |
| INFANTS | | | | | |
| 0-<3 | mo | 10-12% | 120 | 150-125 | 35% |
| 3-8 | mo | 10-12% | 115 | 160-130 | 35% |
| 6-9 | mo | 10-12% | 110 | 145-125 | 35% |
| 9-12 | mo | 10-12% | 105 | 135-120 | 35% |
| | | g/kg | kcal/kg/day | mL/day | % Kal/d |
| Children | | | | | |
| 1-3 | years | 2-2.8 | 102 | 900-1800 | 35% |
| 4-6 | years | 2 | 90 | 1300-2300 | 35% |
| 7-10 | years | 1.5 | 70 | 1650-3300 | 35% |
| WOMEN | | | | | |
| 11-14 | years | 1 | 47 | 1500-3000 | 35% |
| 15-18 | years | 0.8 | 40 | 2100-3000 | 35% |
| >18 | years | 0.8 | 20-25 | 1400-2500 | 35% |
| MEN | | | | | |
| 11-14 | years | 1 | 55 | 2000-3700 | 35% |
| 15-18 | years | 0.9 | 45 | 2100-3900 | 35% |
| >18 | years | 0.8 | 20-25 | 2000-3300 | 35% |

If patient is >20% ideal body weight (IBW), use upper range IBW to calculate needs

Since the recognition of phenylketonuria and the development of the successful phenylalanine-restricted diet by Dr Horst Bickel, treatment of many inborn errors of metabolism has involved restriction of the dietary precursor to the affected pathway. This has been true for decades and is still the mainstay for therapy of disorders affecting mitochondrial β-oxidation and defects in the branched-chain amino acid pathways. The ‘toxicity’ associated with many of these disorders has been thought to result from the accumulation of abnormal chemical intermediates as a result of the enzyme deficiency. While, in some disorders, this may in fact play a role in the pathogenesis, the loss of energy metabolites due to these catabolic disorders has not been systematically evaluated as a potential common contributor to the pathogenesis. This review examines the potential effects of removing a major dietary source (such as fatty acids or glycogen/carbohydrates) from the energy production required for normal metabolic homeostasis. This perspective led to a consideration of the impact of these disorders on the functioning of the citric acid cycle (CAC) and the transfer of important energy-producing IS compounds within and between organs. This exercise resulted in a new focus on ‘anaplerosis’ or ‘filling up’ of the CAC for the purpose of providing an alternative source of energy (Mochel, et al., 2005; Roe, et al., 2002). The experience with the anaplerotic compound triheptanoin, a triglyceride with odd-numbered fatty acids (heptanoate) will be reviewed. A proposed relationship between the metabolism of heptanoate and regulation of intermediary metabolism (catabolic vs anabolic pathways) through the action of ‘nutrient sensors’ (such as AMP-mediated protein kinase (AMPK) and the mammalian target of rapamycin (mTOR)) will also be discussed.

Beginning with phenylketonuria, dietary therapy for inborn errors has focused primarily on the restriction of the precursor to an affected catabolic pathway in an attempt to limit the production of potential toxins. Anaplerotic therapy is based on the concept that there may exist an energy deficit in these diseases that might be improved by providing alternative substrate for both the citric acid cycle (CAC) and the electron transport chain for enhanced ATP production. This article focuses on this basic problem, as it may relate to most catabolic disorders, and provides our current experience involving inherited diseases of mitochondrial fat oxidation, glycogen storage, and pyruvate metabolism using the anaplerotic compound triheptanoin. The observations have led to a realization that ‘inter-organ’ signalling and ‘nutrient sensors’ such as adenylate monophosphate mediated protein kinase (AMPK) and mTOR (mammalian target of rapamycin) appear to play a significant role in the intermediary metabolism of these diseases. Activated AMPK turns on catabolic pathways to augment ATP production While turning off synthetic pathways that consume ATP. Information is provided regarding the inter-organ requirements for more normal metabolic function during crisis and how anaplerotic therapy using triheptanoin, as a direct source of substrate to the CAC for energy production, appears to be a more successful approach to an improved quality of life for these patients.

Methods. Blood acylcarnitine and urinary organic acid analyses have been described previously (Rasped, et al., 1997; Sweetman 1991). Quantitative analysis of amino acids in plasma was determined by ion-exchange HPLC with post-column derivatization using ninhydrin. The amino acids were detected by UV-vis at 570 nm and data integration was performed with PeakNet software version 6.30 (Dionex, Sunnyvale, Calif., USA) (Macchi, et al., 2000).

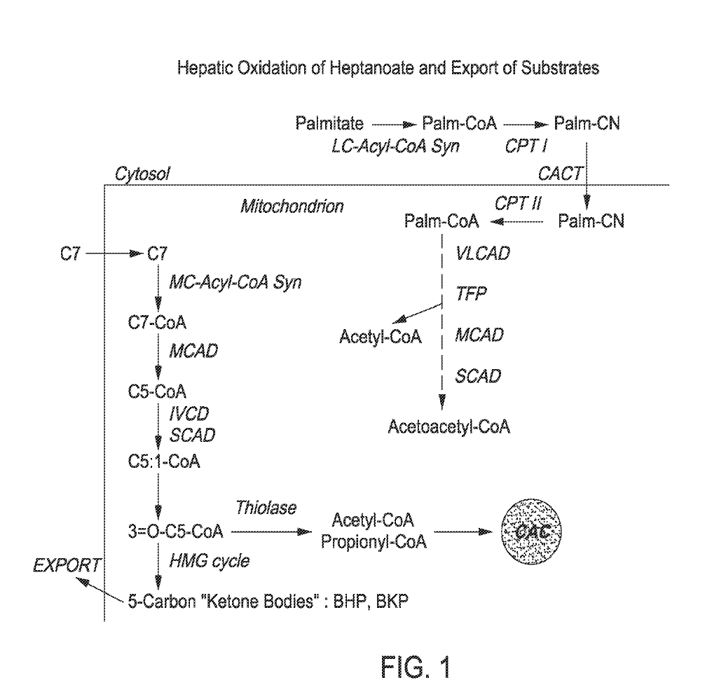

Clinical experience with triheptanoin. Metabolism of triheptanoin when ingested, one mole of triheptanoin is split into one mole of glycerol and 3 moles of heptanoic acid that are metabolized mainly in liver. FIG. 1 summarizes the oxidation of heptanoate (C₇) and the export of 5-carbon ketone bodies that are also produced in the liver. C₇ can enter the mitochondrion largely as a carboxylate, but it is possible that it may also undergo cytosolic activation and then be exchanged for carnitine, as occurs with other longer chain-length fatty acids. The fact that C₇ does not require CPT I, carnitine-acylcamitine translocase or CPT II for entry and oxidation, suggests that it largely enters the mitochondrion as a carboxylate. Presumably, it is converted to C7-CoA by a medium-chain acyl-CoA synthetase and undergoes a cycle of β-oxidation to pentanoyl-CoA (C₅-CoA), which requires the medium-chain acyl-CoA dehydrogenase (MCAD). Pentanoyl-CoA (N-valeryI-CoA) can be used as substrate by isovaleryl-CoA dehydrogenase, which permits oxidation even in the absence of the short-chain acyl-CoA dehydrogenase (SCAD). The partial cycle of β-oxidation produces β-ketopentanoyl-CoA (BKP-CoA), which can be cleaved by thiolase to provide acetyl-CoA and propionyl-CoA to fuel the hepatic CAC. For propionyl-CoA to enter the CAC as succinyl-CoA, both propionyl-CoA carboxylase and methylmalonyl-CoA mutase must be unimpaired. Dietary triheptanoin could be detrimental in disorders such as propionic acidaemia or methylmalonic aciduria since entry into the CAC would be blocked. β-Ketopentanoyl-CoA can also proceed through the HMG cycle, resulting in export of the 5-carbon ketone bodies β-ketopentanoate (BKP) and β-hydroxypentanoate (BHP). When the enzymes of ketone utilization are intact, BKP and BHP serve as substrates to the CAC in other organs, such as muscle, kidney, heart and brain. To date, the experience with triheptanoin in each of the defects of mitochondrial β-oxidation (excluding MCAD deficiency), pyruvate carboxylase deficiency (type B) and adult-onset acid maltase deficiency (GSD II). The following descriptions are highlights of these studies.

Mitochondrial β-oxidation. When triheptanoin represents 30-35% of total caloric intake in VLCAD-deficient patients, hypertrophic cardiomyopathy, congestive heart failure, hepatomegaly and muscle weakness were all relieved. Rhabdomyolysis following infection was not prevented, but the episodes were less frequent and less severe (Roe, et al., 2002). The need to restrict simple carbohydrate in the diet of these patients became apparent with unexpected weight gain when polycose or simple dietary sugars were not reduced in the presence of triheptanoin. A complete summary of the observations with 48 patients with defects of mitochondrial β-oxidation is being prepared for publication elsewhere. The major observations of this experience can be summarized as follows: The patients included were cases of deficiency of CPT I (2), carnitine acylcarnitine translocase (1), CPT II (7), VLCAD (19), LCHAD (9), mitochondrial trifunctional protein (5), and SCAD (5). Each patient was included in a protocol lasting 18 months and each served as their own control comparing prior conventional therapy versus experience with triheptanoin. Following introduction of the diet and education for 5 days, patients were re-evaluated clinically and biochemically at 2, 6 and 12 moths and finally at 18 months. Despite the fact that these investigations were not a crossover double blind study, which should be performed, the overall results suggested some interesting potential benefits to this population, as presented in Table 1.

TABLE 1
Clinical symptoms and results of dietary therapy for fat oxidation disorders

| | | | Rhabdo- | | Weakness/ | | Hypogly- | | Hepato- | | Reti- | |
|---|---|---|---|---|---|---|---|---|---|---|---|---|
| | Cardiac | | myolysis | | fatigue | | caemia | | megaly | | nopathy | |
| Disorder (no.) | Conv.^{a} | C₇^{b} | Conv. | C₇ | Conv. | C₇ | Conv. | C₇ | Conv. | C₇ | Conv. | C₇ |
| CPT1 (2) | 0 | 0 | 0 | 0 | 2 | 0 | 2 | 0 | 2 | 0 | 0 | 0 |
| CACT (1) | Intervened at birth, asymptomatic by 7 months, died with rotavirus | | | | | | | | | | | |
| CPT II (7) | 1 | 0 | 6 | 1 | 7 | 0 | 4 | 0 | 2 | 0 | 0 | 0 |
| VLCAD (19) | 8 | 1 | 18 | 10 | 18 | 3 | 11 | 1 | 13 | 1 | 0 | 0 |
| LCHAD (9) | 0 | 0 | 7 | 1 | 8 | 1 | 4 | 0 | 5 | 1 | 3 | 3 |
| TFP (5) | 1 | | 5 | 3 | 5 | 4 | 1 | 0 | 1 | 0 | 0 | 0 |
| ‘SCAD’ (5) | 0 | 0 | 0 | 0 | 4 | 2 | 2 | 0 | 3 | 0 | 0 | 0 |
| Total (48) | 10 | 1 | 36 | 15 | 44 | 10 | 24 | 1 | 26 | 2 | 3 | 3 |

^{a}Conv = conventional diet (Mct and/or low-fat, high-carbohydrate
^{b}C₇ = heptanoate

When compared to a study using conventional (MCT) diet therapy reported in 1999 (Saudubray, et al., 1999), our current experience with the triheptanoin diet revealed that cardiomyopathy was resolved, hypoglycaemia and hepatomegaly were eliminated, and rhabdomyolysis was less frequent but not eliminated. The peripheral neuropathy of trifunctional protein (TFP) deficiency and the retinopathy seen in some patients with LCHAD deficiency was not improved. Mortality was 6% (3 of 48 patients)—one of which cases (VLCAD) was due to noncompliance with any therapy. The mortality of the earlier study was 21 of 41 patients (51%), which was markedly increased owing to inclusion of 9 patients with neonatal onset of CPT II and translocase (CATR) deficiency, all of whom died. However, in this earlier study with conventional therapy, 6 of 8 VLCAD, all 4 TFP and 2 of 10 LCHAD patients died (12 of 24=50% mortality) compared to 1 of 19, 1 of 5 and 0 of 9, respectively (2 of 23=9% mortality) for patients receiving the triheptanoin diet. These comparisons suggest a possibly reduced mortality rate with the triheptanoin trial for these three defects.

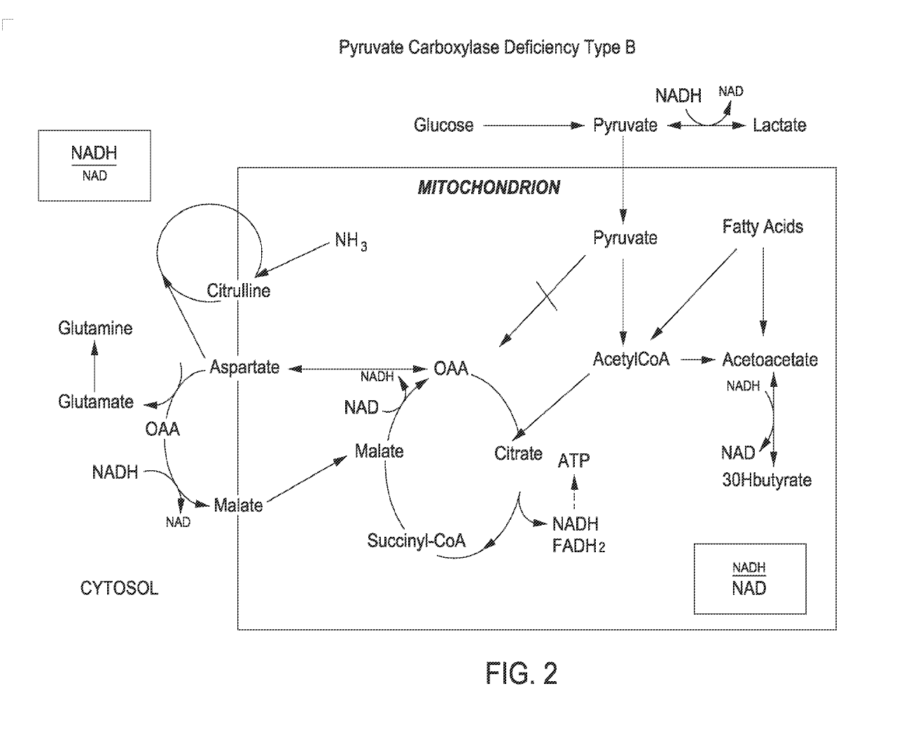

Pyruvate carboxylase (PC) deficiency (type B). The previously reported experience (Mochel, et al., 2005) involved the most severe phenotype, which manifests hepatic failure, severe lactic acidosis, ketoacidosis, and elevated citrullinaemia with hyperammonaemia. The perturbed metabolic scheme is presented in FIG. 2. In the untreated acute episode, the major abnormalities are in the ratio of NADH:NAD, which is increased in the cytosol and facilitates the production of lactate from pyruvate, while it is decreased in the mitochondrial matrix. This apparent reduction in the mitochondrial ratio reflects reduced CAC activity due to lack of substrate as well as the extreme reversal of the ratio of 3-hydroxybutyrate to acetoacetate. From this figure, it can also be deduced from the ketosis that the acyl-CoA:CoASH ratio is also altered, and this is known to impair the activities of pyruvate dehydrogenase, isocitrate dehydrogenase and α-ketoglutarate dehydrogenase in the CAC.

The present invention was used to evaluate the effects of an odd-chain fatty acid based treatment. Enteral intervention with a formula containing 4 grams of triheptanoin per kg body weight (35% of total caloric intake) had an immediate effect within 24 hour on these metabolic derangements. The present inventors have identified the immediate correction of plasma metabolite levels during dietary treatment with triheptanoin. Changes in plasma levels of ammonia (NH₃), citrulline (Cit) and glutamine (Gln) occur during the first 48 hours of triheptanoin diet therapy (see e.g., Mochel, et al., (2005)).

Both the lactate and the lactate:pyruvate ratio decreased rapidly, but not to the normal range—possibly indicating reduced glycolysis and a more normal ratio of cytosolic NADH:NAD (metabolism of the glycerol backbone of triheptanoin would produce pyruvate and thus lactate in this disorder). The redox state in the mitochondrion was similarly relieved, as evidenced by the extreme reversal of the 3-hydroxybutyrate:acetoacetate ratio in only 4 h following administration of enteral triheptanoin. In the same time frame, both citrulline and ammonia decreased. The sudden return to normal citrulline and ammonia levels reflects the increased availability of aspartate to form argininosuccinate. FIG. 2 shows the increased availability of oxaloacetate forming aspartate and facilitating the cytosolic argininosuccinate synthetase reaction. The progressive in crease in the plasma glutamine concentration may represent protein sparing in this situation. It is of special note that, with these very rapid changes, hepatic protein synthesis was stimulated, as evidenced by the complete restoration of normal levels of clotting factors and the elimination of hepatic failure. Also associated with these metabolic corrections was evidence for enhanced γ-aminobutyric acid (GABA) levels in the cerebrospinal fluid. Sequential magnetic resonance imaging in this patient revealed that there was no further development of neurodegenerative lesions while on this diet. The physiological role of 4-carbon ketone bodies in brain metabolism is well recognized (Nehlig, et al., 1993). The 5-carbon ketone bodies, generated and exported by the liver, can fuel the CAC and may have even greater potential value for neurological disorders associated with impaired energy production.

Adult-onset acid maltase deficiency (GSD II). Adult-onset acid maltase deficiency is a lysosomal storage disorder, affecting the degradation of glycogen in muscle, is characterized by a progressive decline in muscle mass and function to the extent that it finally compromises the diaphragm and accessory muscles of respiration, leading to respiratory failure and death. As with PC deficiency, the details of the successful experience with triheptanoin in a single patient will be presented. There are certain facts about this disorder that are not usually considered. The most important, is that lysosomal ‘acid maltase’ actually contains both acid-α-glucosidase and acid-debrancher activity and, therefore, represents a complete degradation system for glycogen in the lysosome (Brown, et al., 1970). Its name is therefore misleading by indicating only ‘acid-α-glucosidase’ activity. This enzyme is present in the lysosomes of all visceral organs. Its absence in striated skeletal muscle is associated with both glycogen storage (lysosomal and cytosolic) and autophagic vacuoles that reflect extreme protein turnover and degradation. What is really difficult to explain is that the absence of this enzyme is equally severe in liver as in muscle and yet storage of glycogen does not occur in liver. Why is the liver spared in the absence of this lysosomal enzyme when the cytosolic glycogen degradation pathway (neutral pH) is apparently unimpaired? (DiMauro, et al., 1978; Van der Walt, et al., 1987).

One possible explanation is that there are certain potentially energy-rich substrates that are imported from other organ systems by the liver and that preserve its metabolic integrity. In patients in the early stages of this disease, both alanine and glutamine levels in plasma are very reduced. This has prompted attempts with high-protein, low-carbohydrate diets as well as supplements of alanine to attempt to correct these abnormalities (Bodamer, et al., 1997, 2000, 2002; Slonim, et al., 1983). Although there have been sporadic reports suggesting potential benefit from these dietary strategies, there have, as yet, been no conclusive studies that are associated with consistent benefit to these patients. Since the low plasma levels of alanine and glutamine seem to be a feature of some patients with adult-onset acid maltase deficiency, the role of these amino acids as potential substrates from organ systems such as skeletal muscle for the benefit of the liver need to be re-evaluated.

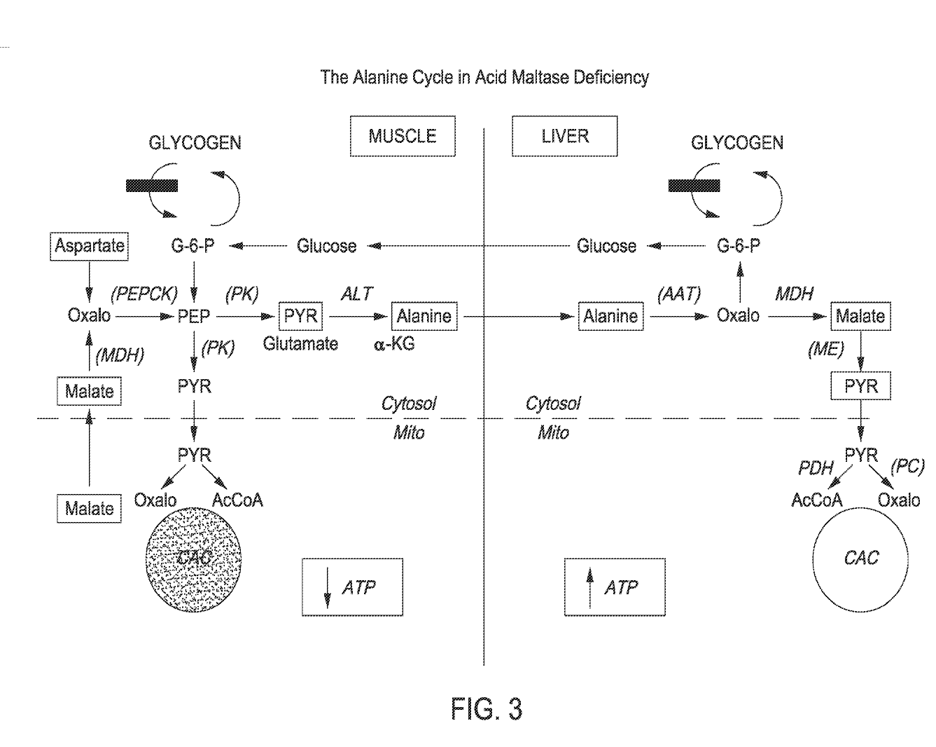

First, the ‘alanine cycle’. It is recognized herein that the ‘alanine cycle’ represents a major contribution from striated skeletal muscle for the preservation of hepatic metabolism, as depicted in FIG. 3. However, it is not without significant cost to the intermediary metabolism of muscle by the diversion of pyruvate to alanine and its export. This can, under conditions of blocked glycogen degradation, result in a ‘steal’ of CAC metabolites (such as pyruvate) from muscle as well as a diversion of needed oxaloacetate from muscle cells to produce the necessary alanine to meet the liver's need for pyruvate. Pyruvate provides both acetyl-CoA and oxaloacetate in liver mitochondria as anaplerotic fuel to the CAC for improving energy production via the electron transport chain. The cost of this transfer of alanine from striated skeletal muscle to liver could be significant and could deprive muscle cells of needed substrate for their own energy support (e.g. malate, pyruvate, oxaloacetate, and α-ketoglutarate). The fact that plasma alanine concentrations are decreased in some patients with this disorder can be interpreted in two ways: (1) not enough is being produced, or (2) what is being produced is being utilized at such a rapid rate that the plasma levels are reduced by rapid hepatic consumption. The alanine cycle is a ‘one-way street’ from muscle to liver with potentially severe consequences for energy metabolism in striated skeletal muscle (Salway 2004).

Decreased plasma glutamine (GLN) concentrations can also be Observed in this disorder. Although it is true that GLN is frequently associated with potential effects in the central nervous system and neurotransmitter synthesis, this association may exclude consideration of the important roles of GLN as a source of energy for many visceral organs, including the liver, as well as a precursor for gluconeogenesis by the kidney. There are some very interesting aspects of glutamine synthesis and its utilization between organ systems for preserving the homeostasis of intermediary metabolism (Curthoys, et al., 1995; Labow, et al., 2001; Watford 2000; Watford, et al., 2002). Unlike most amino acids, both alanine and glutamine are critical for inter-organ metabolic homeostasis. Large quantities of GLN are produced and exported for the benefit of other organs from striated skeletal muscle, and, interestingly, also in large quantities from lung and adipose tissue. The organs that depend on this export and mainly import GLN for energy include liver, kidney, intestine and brain. Once again, the liver needs all of the glutamine that it can obtain to fuel its urea cycle and gluconeogenesis. This relationship between muscle and liver metabolism is supported by a fascinating disparity between the specific activities of certain very important enzymes related to glutamine metabolism that influence its export from certain organ systems, and its utilization by others. In striated skeletal muscle, as in adult-onset acid maltase deficiency, muscle protein is turned over and degraded. The branched-chain aminotransferase (BCAT) in muscle is expressed and active to a much greater extent than in liver. Also, the branched-chain ketoacid dehydrogenase complex (BCKDC), which permits further oxidation for energy purposes in muscle, is much reduced compared to that in liver tissue. The net result will be that amino acids from muscle protein are transaminated, effectively, but are not easily processed for energy production in muscle. The result will be increased export of α-ketoacids from muscle branched-chain amino acid metabolism to the liver, where BCAT is reduced but BCKDC activity is optimal. This would permit complete oxidation and production of energy from the α-ketoacids from muscle branched-chain metabolism as nutritional support to the hepatic CAC (Harris, et al., 2001, 2005). The simultaneous increased production and export of glutamine from muscle protein compromises muscle metabolism but provides important substrate to liver and kidney. This relationship may explain the absence of hypoglycaemia and hyperammonaemia in this disorder. The interaction and interdependency of organ systems for preservation of energy metabolism, in vivo, may be an important consideration in this disease.

With this background, a review of the inventors' experience with the triheptanoin diet in a 42-year-old caucasian female patient with the adult-onset ‘α-glucosidase’ deficiency is relevant. She had a 2-year history of muscle weakness and weight loss associated with impaired respiration that led to respiratory failure. Both plasma alanine and glutamine levels were reduced. Table 2 presents the serial changes in the patient's plasma amino acids when she experienced respiratory failure. On admission, following informed consent, it took only 13 h of dietary triheptanoin to restore her plasma alanine and glutamine to normal levels.

Table 2 shows the return to normal plasma levels of all amino acids during treatment.

| | | Ad- | | | | | | |
|---|---|---|---|---|---|---|---|---|
| | | mis- | | | | | | |
| | | sion | C₇ = | | | | C₇ = | |
| | | base- | 1.0 g/kg | | NPO^{a} | | 1.5 g/kg | |
| | | line | 13 h | 41 h | 65 h | 84 h | 108 h | 132 h |
| Ala- | (162-572) | 129 | 551 | 450 | 189 | 184 | 307 | 277 |
| nine | | | | | | | | |
| Gluta- | (424-720) | 430 | 827 | 424 | 313 | 483 | 584 | 519 |
| mine | | | | | | | | |
| ^{b}Leu- | (60-204) | 104 | 154 | 266 | 158 | 108 | 238 | 220 |
| cine | | | | | | | | |
| ^{b}Val- | (108-295) | 188 | 333 | 384 | 200 | 183 | 304 | 269 |
| ine | | | | | | | | |
| ^{b}Iso- | (39-119) | 58 | 81 | 138 | 87 | 61 | 144 | 128 |
| leu- | | | | | | | | |
| cine | | | | | | | | |
| Total | (1540-4415) | 1162 | 2498 | 2333 | 1246 | 1276 | 2072 | 1843 |
| AA^{c} | | | | | | | | |

^{a}NPO for gastrostomy, i.v. glucose only.
^{b}Essential amino acids.
^{c}AA, amino acids.

While she was waiting for gastrostomy placement but without triheptanoin diet supplement (NPO), her plasma amino acids levels decreased rapidly to admission levels. Following placement of the gastrostomy tube and resumption of the diet, all levels rapidly returned to normal levels. These responses suggest that triheptanoin spares protein turnover in this disorder. This patient returned to a normal lifestyle, gained weight (muscle mass) from 45.3 kg to 56.4 kg, returned to full-time work and has not been affected by her disorder for more than 2 years while receiving this therapy.

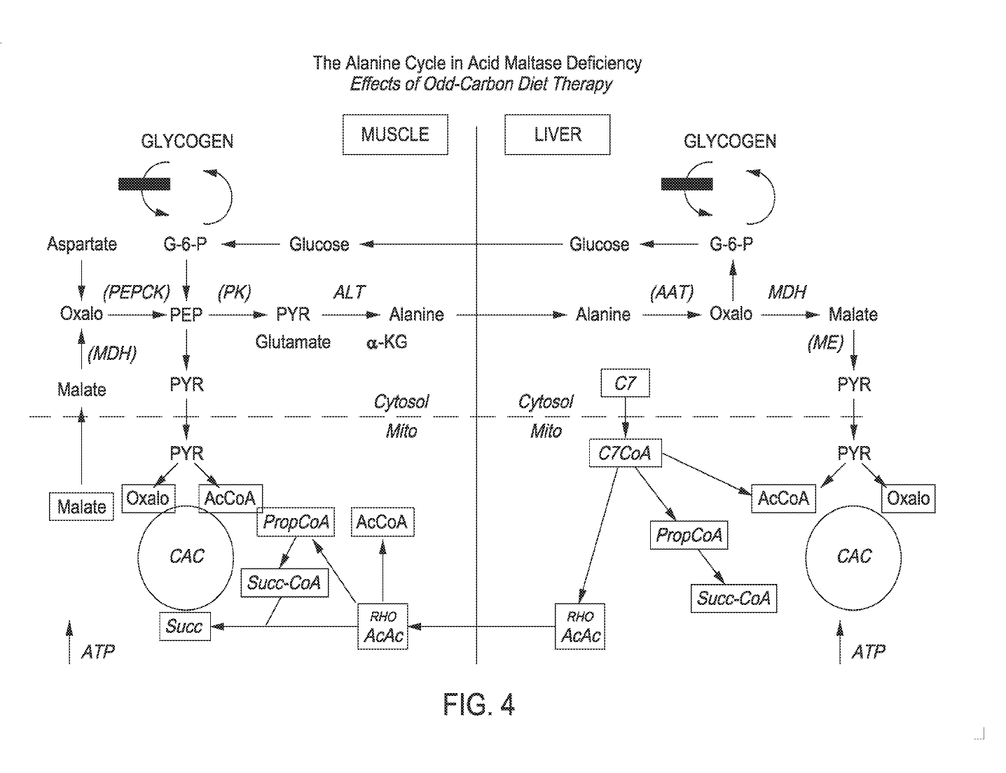

FIG. 4 illustrates how heptanoate metabolism fuels the CAC of liver and how the export of the 5-carbon ketone bodies (BKP and BHP) offset the energy deficit in muscle. This clinical response is unprecedented for this disorder and was independent of enzyme replacement therapy, for which she had been excluded. These observations suggest that triheptanoin diet therapy can provide the needed (anaplerotic) fuel for the CAC, in multiple organs, and may compensate for the energy deficiency that may be associated with so many inherited diseases involving catabolic pathways. Adult-onset acid maltase deficiency appears to emphasize the importance of this exchange of ‘nutrients’ between organ systems.

Nutrient sensors and the relationship to inherited disorders, it is a key recognition of the present invention that biochemists have failed to evaluate the potential role of ‘nutrient sensors’ such as AMP-mediated protein kinase (AMPK) and mTOR and how they might influence the pathology and inconsistent therapeutic benefit for our patients. It is found herein that the role of AMPK is extremely interesting as it relates to disorders affecting catabolic pathways such as fat oxidation disorders, branched-chain amino acid (BCAA) disorders, the glycogenoses and possibly many other disorders. AMPK is a ‘nutrient sensor’ that senses changes in cellular levels of AMP relative to ATP. It is a protein kinase that places PO₄ on serine residues in many proteins, including enzymes (Hardie 2003). The phosphorylation inactivates those enzyme proteins. Since so many enzymes are either activated or inactivated as a result of phosphorylation/dephosphorylation, these mechanisms can have a profound effect on intermediary metabolism. In situations where the availability of ATP is reduced relative to AMP, AMPK is activated. This may result from either decreased ATP production or increased ATP consumption. Either mechanism raises the AMP:ATP ratio. A relative reduction in ATP production would seem to be a reasonable consequence of an impaired catabolic pathway designed to produce ATP—as in an inborn error affecting catabolism. The relative increase of AMP would then activate AMPK. Conversely, if catabolic pathways are intact and ATP production is stimulated, then the subsequent decrease of AMP relative to ATP would inactivate the AMPK. When AMPK is activated, it inactivates those enzymes involved in ‘synthesis’ (anabolism) and activates those that are involved in ‘degradation’ (catabolism) in an attempt to provide more ATP. From the point of view of inherited catabolic defects, this means that all systems that will produce ATP are turned on and those systems (synthetic) that consume ATP are shut down. This may not always be beneficial when a pathway is impaired (e.g. by a long-chain fatty acid oxidation disorder). In that setting, enhanced lipolysis with impaired β-oxidation could increase the production of potentially toxic metabolites. Reversal of this potentially dangerous result of activation of AMPK would require an alternative source of CAC substrate and secondary in crease in ATP. This is the underlying concept of anaplerotic therapy and the reasonable benefit expected from dietary triheptanoin.

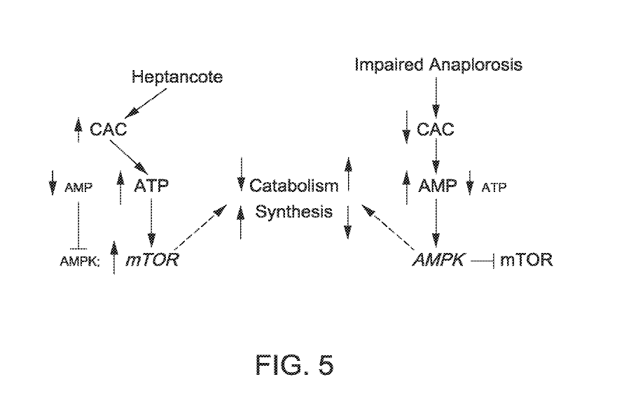

In association with the effects of AMPK, there is another ‘nutrient sensor’ that needs to be considered, called ‘mTOR’ (mammalian target of rapamycin) (Fingar and Blenis 2004). This is also a serine-threonine kinase that has dramatic influence on protein synthesis and cell proliferation. The present invention takes advantage of the very special interaction with AMPK (FIG. 5). mTOR is critical for stimulating protein synthesis. Since AMPK and mTOR are interactive, supply of sufficient substrate to the CAC can, in many organs, decrease the AMP:ATP ratio and therefore inactivate AMPK. This removes the inhibition by AMPK of mTOR, allowing mTOR to turn on protein synthesis. The inactivation of AMPK also allows an increase in other ‘synthetic’ processes such as gluconeogenesis and fat synthesis. The goal of treatment strategies for defects of fat oxidation or BCAA disorders (organic acidurias), etc. would be to ‘fuel’ the CAC, compensate for the secondary ‘energy’ deficiency, and thereby relieve the need for endogenous turnover of protein, carbohydrate or fats as sources of energy. For example, in adult-onset acid maltase deficiency, glycogen is an inadequate source of energy—especially in striated skeletal muscle. Muscle biopsies reveal evidence of proteolysis with autophagic vacuoles as well as glycogen storage in lysosomes and cytoplasm. The liver remains normal without glycogen storage or other functional impairments such as hypoglycaemia or hyperammonaemia despite absence of acid maltase. Instead, nutrients derived from muscle protein turnover and other substrates are transferred to the liver for preservation of its functions (alanine, glutamine, the α-ketoacids from BCAAs). The result is loss of muscle mass. endurance and function. Finally, extreme compromise of the muscles of respiration leads to respiratory failure and death. This sequence may represent the activation of AMPK in an attempt to provide more energy (ATP), which allows muscle catabolism to proceed unabated but with simultaneous inhibition of mTOR that results in proteolysis and autophagy and impaired protein synthesis. Fuelling the CAC with triheptanoin may have reversed this scenario by altering the AMP:ATP ratio from its metabolism, thus inactivating AMPK and activating mTOR with the resultant cessation of all symptoms and associated muscle mass increase (protein synthesis).

Pyruvate carboxylase deficiency (type B). Important features of this disorder include lactic acidosis (increased cytosolic NADH:NAD ratio), ketosis with reduced 3-hydroxybutyrate and extremely increased acetoacetate (decreased mitochondrial NADH:NAD ratio), increased citrulline and ammonia, and hepatic failure with decreased clotting factors, etc. (impaired protein synthesis), in this disorder, the sources of acetyl-CoA and oxaloacetate needed to ‘prime’ the CAC are severely compromised. The CAC needs an alternative source of substrate under this severe restriction. The ‘nutrient sensors’ can respond by enhancing ‘degradation’ of carbohydrate, β-oxidation of fatty acids (ketogenesis), and catabolism of amino acids by enhanced proteolysis to serve as substrate to the CAC in an attempt to increase ATP production. Direct fuelling of the CAC from the metabolism of triheptanoin reversed these abnormalities within 24 h. The ratios of NADH:NAD were reversed, lactate decreased, stimulation of the CAC with substrate provided adequate oxaloacetate for conversion to aspartate to facilitate the reaction of citrulline to argininosuccinate, and the ammonia level decreased. All of these changes suggest that the alteration of the AMP:ATP ratio due to triheptanoin metabolism shut down AMPK and its inhibition of mTOR, thus stimulating synthetic pathways (including protein synthesis).

It was found that these interactions between nutrient sensors can have a profound impact on the management of mitochondrial long-chain fatty acid disorders. The clinical results of the triheptanoin trial in those disorders seem to follow the same principles—as observed by elimination of hypoglycaemia and hepatomegaly, reversal of cardiomyopathy, and elimination of the decreased muscle endurance and strength.

Triheptanoin is not the only potentially useful anaplerotic agent, but it illustrates the potential benefit of focusing on the ‘terminal pathways’ and providing substrate to the CAC when there is an associated reduction in energy-rich substrate due to the inherited biochemical defect. The primary purpose in presenting these data is to stimulate consideration of scientific information that has not been applied to the therapy of inherited metabolic disease—namely the consequences of diminished energy production as it relates to the fuelling of the CAC and the subsequent considerations that relate to ‘nutrient sensors’. As physicians and scientists whose activities are oriented towards identifying new problems in biochemical genetics, our hope is to provide alternative concepts for improving the quality of life of our patients. With that in mind, we must continue to search for new strategies that might fulfill this goal. Restriction of dietary precursors has not been uniformly effective, though palliative. Perhaps we have not been sufficiently focused on the ‘secondary’ consequences that may be more significant impediments to a normal lifestyle for these patients. Since, at this time, we have been the only group to observe many of the surprising effects of anaplerotic therapy, it would be very useful if others explored this potentially beneficial strategy. With the current limitations that exist in the development of consistently beneficial enzyme or gene replacement therapies, anaplerotic diet therapy may be a timely alternative.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Bodamer O A, Haas D, Hermans M M, Reuser A J, Hoffman G F (2002) L-Alanine supplementation in late infantile glycogen storage disease type II. Pediatr Neurol 27: 145-146.

Bodamer O A, Leonard J V, Halliday D (1997) Dietary treatment in late onset acid maltase deficiency. Eur J Pediatr 156(Supplement 1) 528 S39-42.

Bodamer O A, Halliday D, Leonard J V (2000) The effects of L-alanine supplementation in late-onset glycogen storage disease type II. 531 Neurology 55: 710-712.

Brown B I, Brown D H, Jeffrey P L (1970) Simultaneous absence of alpha-1,4-glucosidase and alpha-1,6-glucosidase activities (pH 4) in tissues of children with type II glycogen storage disease. Biochemistry 9(6): 1423.-1428.

Curthoys N P, Watford M (1995) Regulation of glutaminase activity and glutamine metabolism. Annu Rev Nutr 15: 133-159.

DiMauro S, Stem L Z, Mehler M, Nagle R B, Payne C (1978) Adult onset acid maltase deficiency: a postmortem study. Muscle Nerve 1: 27-36.

Fingar D C, Blenis J (2004) Target of rapamycin (TOR): an integrator of nutrient and growth factor signals and coordinator of cell growth and cell cycle progression. Oncogene 23: 3151-3171.

Hardie D G (2003) Minireview: The AMP-activated protein kinase cascade: the key sensor of cellular energy status. Endocrinology 144(12): 5179-5183.

Harris R A, Kobayashi R, Murakami T, Shimomura Y (2001) Regulation of branched-chain α-keto acid dehydrogenase kinase expression in rat liver. J Nutr 131: 841S-845S.

Harris R A, Joshi M, Jeoung N H, Obayashi M (2005) Overview of the molecular and biochemical basis of branched-chain amino acid catabolism. J Nutr 135(6 Supplement): 15275-1530S.

Labow B I, Souba W W Abcouwer S F (2001) Mechanisms governing the expression of the enzymes of glutamine metabolism-glutaminase and glutamine synthetase. J Nutr 131(9 Supplement): 2467S-2474S.

Macchi F D, Shen F J, Keck R G, Harris R J (2000) Amino acid analysis, using postcolumn ninhydrin detection, in a biotechnology laboratory. Methods Mol Biol. 159: 9-30.

Mochel F, deLonlay P, Touati G, et al., (2005) Pyruvate carboxylase deficiency: immediate clinical and biochemical improvement with dietary triheptanoin. Mol Gen Metab 84: 305-312.

Nehlig A, Pereira de Vasconcelos A (1993) Glucose and ketone utilization by the brain of neonatal rats. Prog Neurobiol 40(2): 163-221.

Rashed M S, Bucknall M P, Little D, et al., (1997) Screening blood spots for inborn errors of metabolism by electrospray tandem mass spectrometry with a microplate batch process and a computer algorithm for automated flagging of abnormal profiles. Clin Chem 43: 1129-1141.

Roe C R, Sweetman L, Roe D S, David F, Brunengraber H (2002) Effective dietary treatment of cardiomyopathy and rhabdomyolysis in long-chain fat oxidation disorders using an anaplerotic odd-chain triglyceride. J Clin Invest 110(2): 259-269.

Salway J G (2004) Metabolism at a Glance, 3rd edn. Oxford: Blackwell.

Saudubray J M, Martin D, De Lonlay P. et al., (1999) Recognition and management of fatty acid oxidation defects: a series of 107 patients. J Inherit Mctah Dis 22: 488-502.

Slonim A L, Coleman R A, McElligot M A, et al., (1983) Improvement of muscle function in acid maltase deficiency by high-protein therapy. Neurology 33: 34-38.

Sweetman L (1991) Organic acid analysis. In: Hammes F A, ed. Techniques in Diagnostic Human. Biochemical Genetics: A Laboratory Manual. New York: Wiley-Liss, 143-176.

Van der Walt J D, Swash M, Leake J, Cox E L (1987) The pattern of involvement of adult-onset acid maltase deficiency at autopsy. Muscle Nerve 10: 272-281.

Watford M (2000) Glutamine and glutamate metabolism across the liver sinusoid. J Nutr 130(4S Supplement): 983S-987S.

Watford M, Chellaraj V, Ismat A, Brown ID, Raman P (2002) Hepatic glutamine metabolism. Nutrition 18(4): 301-303.

1.-25. (canceled)

26. A method for treating a condition associated with a catabolic defect in a patient comprising
administering an effective amount of an odd carbon fatty acid source to the patient in need of such treatment,
wherein the amount of the odd carbon fatty acid is sufficient to reduce the activation of AMPK in the patient, and thereby reduce cellular catabolism.

27. The method of claim 26, wherein the amount of odd carbon fatty acid is sufficient to increase ATP production, and thereby reduce the intracellular ratio of AMP to ATP.

28. The method of claim 26, wherein the amount of odd carbon fatty acid is sufficient to reduce the inhibition of mTOR by activated AMPK, and thereby stimulate protein synthesis.

29. The method of claim 26, wherein the amount of odd carbon fatty acid is sufficient to reduce the activation of AMPK and thereby allowing an increase in gluconeogenesis or fatty acid synthesis.

30. The method of claim 29, wherein the increase in gluconeogenesis is accompanied by a decrease in glycolysis and wherein the increase in fatty acid synthesis is accompanied by a decrease in lipolysis and beta-oxidation.

31. The method of claim 26, wherein the amount of odd carbon fatty acid is sufficient to reduce the activation of AMPK and thereby reduce endogenous turnover of protein, carbohydrate or lipid as sources of energy in the patient. The method of claim 26, wherein the odd carbon fatty acid source comprises C5 or C7 fatty acid or a combination thereof.

32. The method of claim 26, wherein the odd carbon fatty acid source is selected from the group consisting of triheptanoin, tripentanoin, n-heptanoic acid, n-pentanoic acid, a triglyceride comprising n-heptanoic acid or n-pentanoic acid or both, n-heptanoate, and n-pentanoate.

33. The method of claim 26, wherein the odd carbon fatty acid source is triheptanoin or tripentanoin or a combination thereof.

34. The method of claim 26, wherein the amount of odd carbon fatty acid corresponds to 30-35% of daily dietary caloric requirement for the patient.

35. The method of claim 26, wherein the administration comprises administering C7 fatty acid source at 1-4 g/kg for infants, 1-2 g/kg for young children, or 0.1-2 g/kg for adult.

36. The method of claim 26, wherein the catabolic defect is caused by an inborn error of metabolism.
